(19) United States
(12) Patent Application Publication
(10) Pub. No.: US 2008/0221399 A1
(43) Pub. Date: Sep. 11, 2008

(54) MONITOR FOR MEASURING VITAL SIGNS AND RENDERING VIDEO IMAGES

(75) Inventors: Zhou Zhou, La Jolla, CA (US); Marshal Singh Dhillon, San Diego, CA (US); Henk Visser, San Diego, CA (US); Matthew John Banet, Del Mar, CA (US); Andrew Stanley Terry, San Diego, CA (US); Kenneth Robert Hunt, Vista, CA (US); Adam Michael Fleming, New York, NY (US)

Correspondence Address: Triage Wireless, Inc.;Matthew John Banet, 9444 Waples Street, Suite 280, SAN DIEGO, CA, 92121, US

(73) Assignee: TRIAGE WIRELESS, INC., San Diego, CA (US)

(21) Appl. No.: 11/682,177

(22) Filed: Mar. 5, 2007

(51) Int. Cl. A61B 5/00

(52) U.S. Cl. 600/301

(57) ABSTRACT

The invention features a vital sign monitor that includes: 1) a sensor component that attaches to the patient and features an optical sensor and an electrical sensor that measure, respectively a first and second signal: and 2) a control component. The control component features: 1) an analog-to-digital converter configured to convert the first signal and second signal into, respectively, a first digital signal and a second digital signal; 2) a CPU configured to operate an algorithm that generates a blood pressure value by processing with an algorithm the first digital signal and second digital signal; 3) a display element; 4) a graphical user interface generated by computer code operating on the CPU and configured to render on the display element the blood pressure value; and 5) a software component that renders video images on the display element. To capture video and audio information, the device further includes both a digital camera and a microphone.

### CROSS REFERENCES TO RELATED APPLICATION

Not Applicable

### STATEMENT REGARDING FEDERALLY SPONSORED RESEARCH OR DEVELOPMENT

Not Applicable

### BACKGROUND OF THE INVENTION

1. Field of the Invention

The present invention relates to monitors for measuring vital signs, e.g. blood pressure, and rendering video images.

2. Description of the Related Art

Pulse transit time (‘PTT’), defined as the transit time for a pressure pulse launched by a heartbeat in a patient's arterial system, has been shown in a number of studies to correlate to both systolic and diastolic blood pressure. In these studies, PTT is typically measured with a conventional vital signs monitor that includes separate modules to determine both an electrocardiogram (‘ECG’) and pulse oximetry. During a PTT measurement, multiple electrodes typically attach to a patient's chest to determine a time-dependent ECG characterized by a sharp spike called the ‘QRS complex’. This feature indicates an initial depolarization of ventricles within the heart and, informally, marks the beginning of the heartbeat and a pressure pulse that follows. Pulse oximetry is typically measured with a bandage or clothespin-shaped sensor that attaches to a patient's finger, and includes optical systems operating in both the red and infrared spectral regions. A photodetector measures radiation emitted from the optical systems and transmitted through the patient's finger. Other body sites, e.g., the ear, forehead, and nose, can also be used in place of the finger. During a measurement a microprocessor analyses red and infrared radiation measured by the photodetector to determine the patient's blood oxygen saturation level and a time-dependent waveform called a plethysmograph. Time-dependent features of the plethysmograph indicate both pulse rate and a volumetric change in an underlying artery (e.g., in the finger) caused by the propagating pressure pulse.

Typical PTT measurements determine the time separating a maximum point on the QRS complex (indicating the peak of ventricular depolarization) and a foot of the plethysmograph (indicating initiation of the pressure pulse). PTT depends primarily on arterial compliance, the propagation distance of the pressure pulse (closely approximated by the patient's arm length), and blood pressure. For a given patient, PTT typically decreases with an increase in blood pressure and a decrease in arterial compliance. Arterial compliance, in turn, typically decreases with age.

A number of issued U.S. patents describe the relationship between PTT and blood pressure. For example, U.S. Pat. Nos. 5,316,008; 5,857,975; 5,865,755; and 5,649,543 each describe an apparatus that includes conventional sensors that measure an ECG and plethysmograph, which are then processed to determine PTT.

Studies have also shown that a property called vascular transit time (‘VTT’), defined as the time separating two plethysmographs measured from different locations on a patient, can correlate to blood pressure. Alternatively, VTT can be determined from the time separating other time-dependent signals measured from a patient, such as those measured with acoustic or pressure sensors. A study that investigates the correlation between VTT and blood pressure is described, for example, in ‘Evaluation of blood pressure changes using vascular transit time’, Physiol. Meas. 27, 685-694 (2006). U.S. Pat. Nos. 6,511,436; 6,599,251; and 6,723,054 each describe an apparatus that includes a pair of optical or pressure sensors, each sensitive to a propagating pressure pulse, that measure VTT. As described in these patents, a microprocessor associated with the apparatus processes the VTT value to estimate blood pressure.

In order to accurately measure blood pressure, both PTT and VTT measurements typically require a ‘calibration’ consisting of one and more conventional blood pressure measurements made simultaneously with the PTT or VTT measurement. The calibration accounts for patient-to-patient variation in arterial properties (e.g., stiffness and size). Calibration measurements are typically made with an auscultatory technique (e.g., using a pneumatic cuff and stethoscope) at the beginning of the PTT or VTT measurement; these measurements can be repeated if and when the patient undergoes any change that may affect their physiological state.

Other efforts have attempted to use a calibration along with other properties of the plethysmograph to measure blood pressure. For example, U.S. Pat. No. 6,616,613 describes a technique wherein a second derivative is taken from a plethysmograph measured from the patient's ear or finger. Properties from the second derivative are then extracted and used with calibration information to estimate the patient's blood pressure. In a related study, described in ‘Assessment of Vasoactive Agents and Vascular Aging by the Second Derivative of Photoplethysmogram Waveform’, Hypertension. 32, 365-370 (1998), the second derivative of the plethysmograph is analyzed to estimate the patient's ‘vascular age’ which is related to the patient's biological age and vascular properties.

A number of patents describe ‘telemedicine’ systems that collect vital signs, such as blood pressure, heart rate, pulse oximetry, respiratory rate, and temperature, from a patient, and then transmit them through a wired or wireless link to a host computer system. Representative U.S. patents include U.S. Pat. Nos. 6,416,471; 6,381,577; and 6,112,224. Some telemedicine systems, such as that described in U.S. Pat. No. 7,185,282, include separate video systems that collect and send video images of the patient along with the vital signs to the host computer system. In these systems separate monitors are typically used to measure vital signs and video images from the patient.

### SUMMARY OF THE INVENTION

The present invention provides a portable patient monitor that measures vital signs (e.g. blood pressure) and renders video images on a high-resolution display. The video images, for example, can be images of the patient sent within or outside of the hospital. Alternatively, the images can be of family members or medical professionals sent to the patient. In both cases, the same monitor used to measure and display the patient's vital signs also collects and renders the video images.

The monitor measures one of the most important vital signs, blood pressure, with a cuffless, PTT-based measurement. Other vital signs, such as heart rate, pulse oximetry, respiratory rate, and temperature, are also measured. In addition, the monitor includes a microprocessor that engages a digital video recording camera, similar to a conventional ‘web-camera’, and a small digital audio microphone to record audio information. In general, the monitor additionally includes many features of a conventional personal digital assistant (‘PDA’), such as a portable form factor, touchpanel, and an icon-driven graphical user interface (‘GUI’) rendered on a color, liquid crystal display (‘LCD’). These features allow a user, preferably a healthcare professional or patient, to select different measurement modes, such as continuous, one-time, and 24-hour ambulatory modes, by simply tapping a stylus on an icon within the GUI. The monitor also includes several other hardware features commonly found in PDAs, such as short-range (e.g., Bluetooth® and WiFi®) and long-range (e.g., CDMA, GSM, IDEN) wireless modems, global positioning system (‘GPS’), digital camera, and barcode scanner.

The monitor makes cuffless blood pressure measurements using a sensor pad that includes small-scale optical and electrical sensors. The sensor pad typically attaches to a patient's arm, just below their bicep muscle. A flexible nylon armband supports the sensor pad and has a form factor similar to a conventional wrap-around bandage. The sensor pad connects to a secondary electrode attached to the patient's chest. During operation, the sensor pad and secondary electrode measure, respectively, time-dependent optical and electrical waveforms that the microprocessor then analyzes as described in detail below to determine blood pressure and other vital signs. In this way, the sensor pad and secondary electrode replace a conventional cuff to make a rapid measurement of blood pressure with little or no discomfort to the patient.

Specifically, in one aspect, the invention features a vital sign monitor that includes: 1) a sensor component that attaches to the patient and features an optical sensor and an electrical sensor that measure, respectively a first and second signal: and 2) a control component. The control component features: 1) an analog-to-digital converter configured to convert the first signal and second signal into, respectively, a first digital signal and a second digital signal; 2) a CPU configured to operate an algorithm that generates a blood pressure value by processing with an algorithm the first digital signal and second digital signal; 3) a display element; 4) a graphical user interface generated by computer code operating on the CPU and configured to render on the display element the blood pressure value; and 5) a software component that renders video images on the display element. To capture video and audio information, the device further includes both a digital camera and a microphone.

The monitor can include removable memory components for storing and transporting information. For example, these components can be a flash component or a synchronous dynamic random access memory (SDRAM) packaged in a removable module. The monitor can communicate with external devices through wireless modems that operate both short-range and long-range wireless protocols. Specifically, these modems may operate on: 1) a wide-area wireless network based on protocols such as CDMA, GSM, or IDEN; and, 2) a local-area wireless network based on protocols such as 802.11, 802.15, or 802.15.4. These protocols allow the monitor to communicate with an external computer, database, or in-hospital information system.

In embodiments, to generate the optical signal, an optical sensor within the sensor pad irradiates a first region with a light source (e.g. an LED), and then detects radiation reflected from this region with a photodetector. The signal from the photodetector passes to an analog-to-digital converter, where it is digitized so that it can be analyzed with the microprocessor. The analog-to-digital converter can be integrated directly into the microprocessor, or can be a stand-alone circuit component. Typically, in order to operate in a reflection-mode geometry, the radiation from the light source has a wavelength in a ‘green’ spectral region, typically between 520 and 590 nm. Alternatively, the radiation can have a wavelength in the infrared spectral region, typically between 800 and 1100 nm. In preferred embodiments the light source and the light detector are included in the same housing or electronic package. In embodiments, an additional optical sensor can be attached to the patient's finger and connected to the sensor pad through a thin wire. This optical sensor can be used to make conventional pulse oximetry measurements, and may additionally measure a plethysmograph that can be analyzed for the blood pressure measurement.

To generate the electrical signal, electrical sensors (e.g. electrodes) within the sensor pad and secondary electrode detect first and second electrical signals. The electrical signals are then processed (e.g. with a multi-stage differential amplifier and band-pass filters) to generate a time-dependent electrical waveform similar to an ECG. The sensor pad typically includes a third electrode, which generates a ground signal or external signal that is further processed to, e.g., reduce noise-related artifacts in the electrical signal.

In embodiments, the electrodes within the sensor pad are typically separated by a distance of at least 2 cm. In other embodiments, the electrodes include an Ag/AgCl material (e.g., an Ag/AgCl paste sintered to a metal contact) and a conductive gel. Typically a first surface of the conductive gel contacts the Ag/AgCl material, while a second surface is temporarily covered with a protective layer. The protective layer prevents the gel from drying out when not in use, and typically has a shelf life of about 24 months. In still other embodiments, the electrodes are made from a conductive material such as conductive rubber, conductive foam, conductive fabric, and metal.

During a measurement, the monitor makes a cuffless, non-calibrated measurement of blood pressure using PTT and a correction that accounts for the patient's arterial properties (e.g., stiffness and size). This correction, referred to herein as a ‘vascular index’ (‘VI’), is calculated according to one of two methods. In the first method, the VI is determined by analyzing the shape of the plethysmograph, measured at either the brachial or the finger artery. In this method, in order to accurately extract features from the shape of the plethysmograph, this waveform is typically first passed through a mathematical filter based on Fourier Transform (called the ‘Windowed-Sinc Digital Filter’) and then analyzed by taking its second derivative. In the second method, the VI is estimated from the VTT measured between the patient's brachial and finger arteries. In both cases, the VI is used in combination with the patient's biological age to estimate their arterial properties. These properties are then used to ‘correct’ PTT and thus calculate blood pressure without the need for an external calibration (e.g., without input of an auscultatory measurement).

The invention has a number of advantages. In general, the monitor combines all the data-analysis features and form factor of a conventional PDA with the monitoring capabilities of a conventional vital sign monitor. This results in an easy-to-use, flexible monitor that performs one-time, continuous, and ambulatory measurements both in and outside of a hospital. And because it lacks a cuff, the monitor measures blood pressure in a simple, rapid, pain-free manner. Measurements can be made throughout the day with little or no inconvenience to the user. Moreover, measurements made with the sensor pad can be wirelessly transmitted to an external monitor. This minimizes the wires connected to the patient, thereby making them more comfortable in a hospital or at-home setting.

These and other advantages are described in detail in the following description, and in the claims.

### BRIEF DESCRIPTION OF THE DRAWINGS

FIG. 1 is a schematic view of a monitor for measuring vital signs and rendering video images according to the invention that connects to a pad sensor on a patient's arm and an electrode on the patient's chest;

FIGS. 2A and 2B show, respectively, front and top views of the monitor of FIG. 1;

FIG. 3A is a schematic top view of the pad sensor of FIG. 1 which includes optical sensors, electrodes, and a clasping arm-band;

FIG. 3B is a schematic top view of a two-piece electrode combined in a non-disposable sensor housing attached to a disposable patch;

FIG. 3C is a schematic top view of a snap connector that connects to the two-piece electrode of FIG. 3B;

FIG. 4 shows a semi-schematic view of multiple body-worn monitors of FIG. 1 connected to a central conferencing system in, e.g., a hospital setting;

FIGS. 5A and 5B show, respectively, bottom and top views of a circuit board within the monitor of FIG. 1;

FIG. 6 shows a schematic view of an embedded software architecture used in the monitor of FIG. 1;

FIGS. 7A and 7B show screen captures taken from a color LCD of FIG. 5B that features an icon-driven GUI; and

FIG. 8 shows a schematic view of an Internet-based system used to send information from the monitor of FIG. 1 to both the Internet and an in-hospital information system.

### DETAILED DESCRIPTION OF THE INVENTION

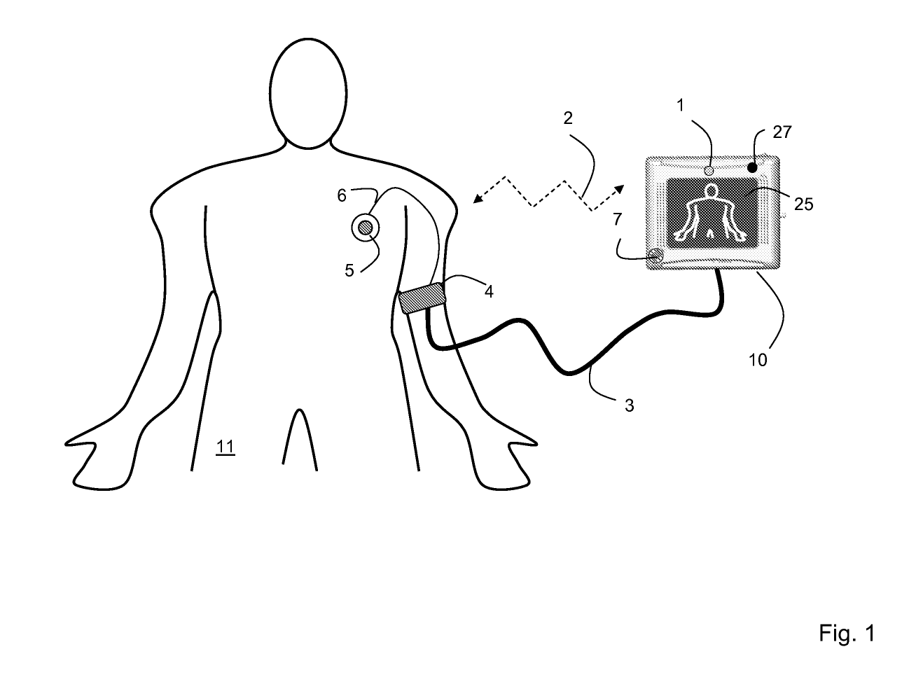

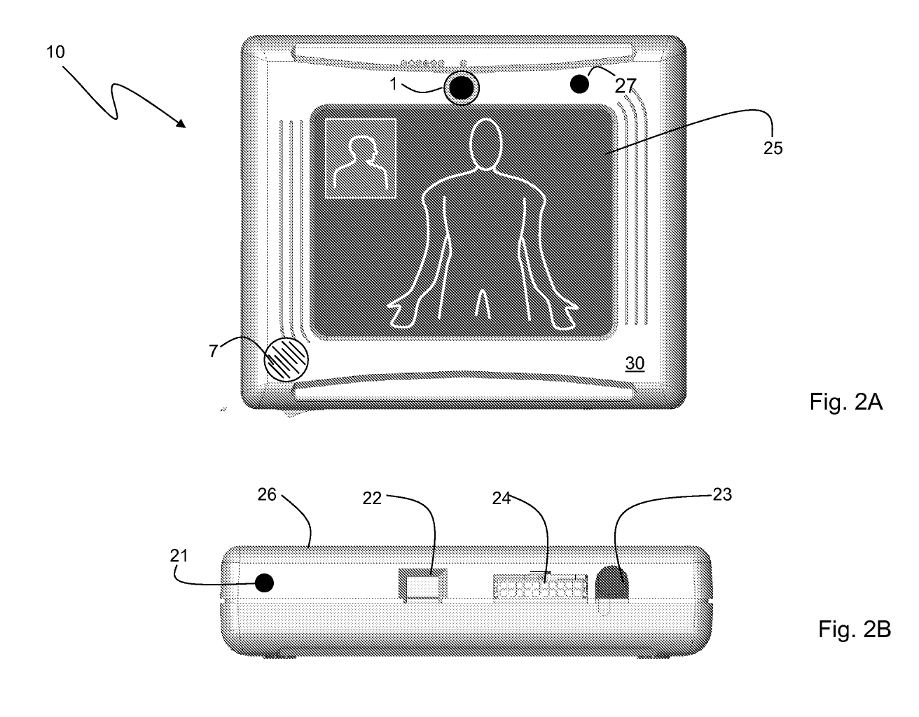

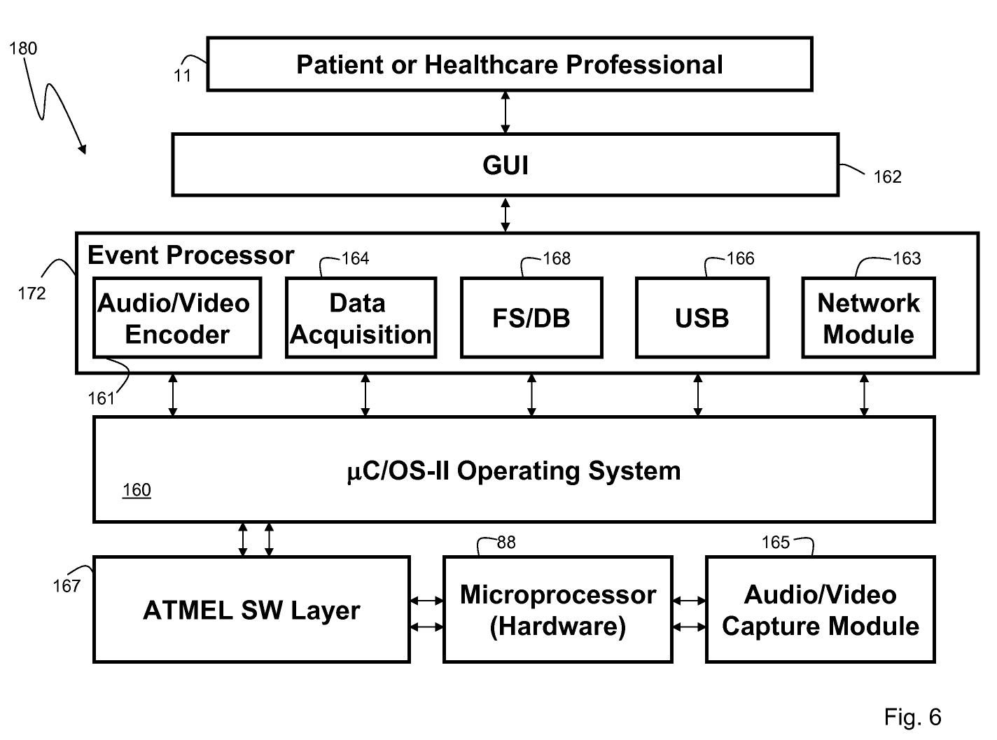

FIGS. 1, 2A, and 2B show a monitor 10 for measuring vital signs and rendering video images according to the invention that features a digital video camera 1, digital audio microphone 27, speaker 7, and GUI rendered on a LCD/touch panel 25. The monitor 10 includes a sensor pad 4 that connects to a patient 11 to measure vital signs such as blood pressure, heart rate, respiratory rate, pulse oximetry, and temperature as described in more detail below. Using the GUI, which is shown in more detail in FIGS. 6A and 6B, and LCD/touch panel 25, a health care professional can activate the digital video camera 1, audio microphone 27, and speaker 7 to exchange audio and video information with the patient through an in-hospital or nationwide wireless network (using, e.g., an antenna 21) or the Internet (using an Ethernet connector, not shown in the figure). In addition, using the GUI the patient can view images of family members during a stay in the hospital. With the same GUI the health care professional can select different vital sign measurement modes, e.g. one-time, continuous, and 24-hour ambulatory mode.

A plastic housing 30 surrounds the monitor 10 to protect its internal components. The monitor 10 additionally includes a barcode reader 22 to optically scan patient information encoded, e.g., on a wrist-worn barcode. A first port 23 receives an external thermometer that measures a patient's esophageal temperature. A second multi-pin port 24 optionally connects to the pad sensor 4 so that these components can connect in a wired mode. The monitor 10 is lightweight by design, and is preferably hand-held to easily position the camera 1 for recording and viewing. In addition, the monitor 10 mounts to stationary objects within the hospital, such as beds and wall-mounted brackets, through mounting holes on its back panel 26.

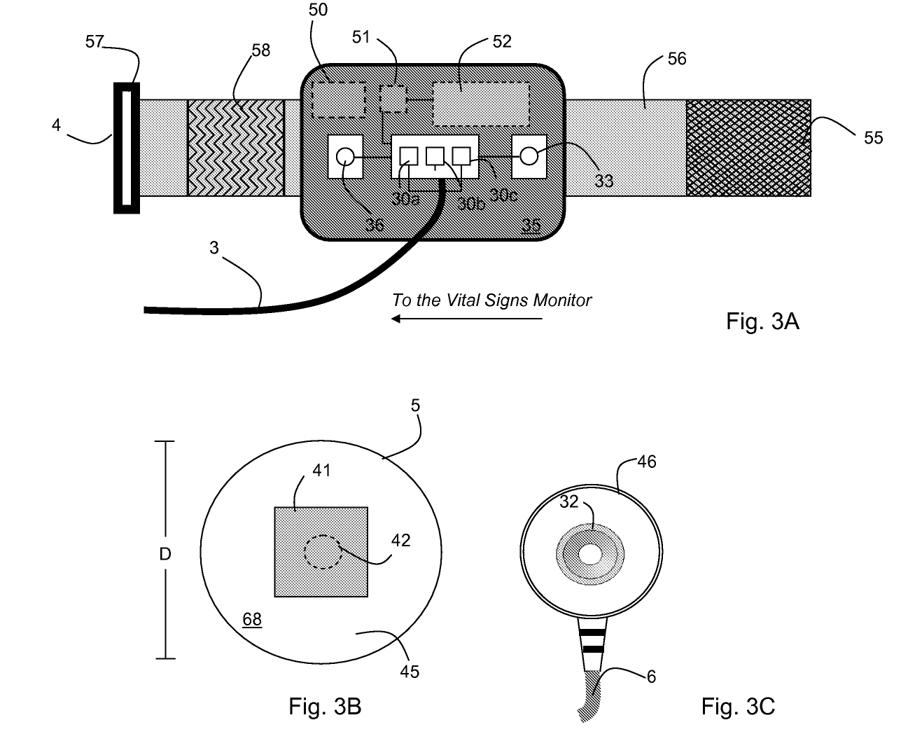

As shown in FIGS. 3A and 3B, the monitor 10 measures vital signs with a pad sensor 4 that attaches to the patient's arm and to a secondary electrode 5. The pad sensor 4 and secondary electrode 5 measure optical and electrical waveforms that are used in an algorithm, described below, to determine blood pressure. During use, the pad sensor 4 wraps around the patient's arm using a VELCRO® belt 56. The belt 56 connects to a nylon backing material 35, which supports three optical sensors 30a-c and two electrodes 36, 33. The belt 56 buckles through a D-ring loop 57 and secures to the patient's arm using VELCRO® patches 55, 58. The pad sensor 4 can connect to the monitor 10 using a coaxial cable 3, or alternatively through a short-range wireless transceiver 50. An analog-to-digital converter 51 within the pad sensor 4 converts analog optical and electrical waveforms to digital ones, which a processor 52 then analyses to determine blood pressure. The secondary electrode 5 connects to the monitor 10 through an electrical lead 6.

To reduce the effects of ambient light, the pad sensor 4 covers the optical sensors 30a-c mounted in the middle of the nylon backing 35. Each optical sensor 30a-c includes light-emitting diodes (LED) that typically emit green radiation (λ=520-570 nm), photodetectors that measure reflected optical radiation which varies in intensity according to blood flow in underlying capillaries, and an internal amplifier. Such a sensor is described in the following co-pending patent application, the entire contents of which are incorporated herein by reference: VITAL SIGN MONITOR FOR CUFFLESSLY MEASURING BLOOD PRESSURE WITHOUT USING AN EXTERNAL CALIBRATION (U.S. Ser. No. 11/______; filed February _{—}, 2007). A preferred optical sensor is model TRS1755 manufactured by TAOS, Inc. of Plano, Tex.

The pad sensor 4 connects to the secondary electrode 5, shown in FIGS. 3B and 3C, which is similar to a conventional ECG electrode. The electrode 5 features a disposable, sterile foam backing 68 that supports an Ag/AgCl-coated male electrical lead 42 in contact with an impedance-matching solid gel 41. An adhesive layer 45 coats the foam backing 68 so that it sticks to the patient's skin. During use, the male electrical lead 42 snaps into a female snap connector 32 attached to a secondary electrode connector 46. The shielded cable 6 connects the secondary electrode 5 to the pad sensor 4 described above. In a preferred embodiment, electrodes 33, 36 measure, respectively, a positive signal and ground signal, while the secondary electrode 5 measures a negative signal. An electrical amplifier in the monitor 10 then processes the positive, negative, and ground signals to generate an electrical waveform, described in detail below, that is similar to a single-lead ECG.

The monitor 10 can also process pulse oximetry measurements typically made by attaching a conventional pulse oximeter sensor to the patient's finger. Determining pulse oximetry in this way is a standard practice known in the art, and is described, for example, in U.S. Pat. No. 4,653,498 to New, Jr. et al., the contents of which are incorporated herein by reference.

In addition to those methods described above, a number of additional methods can be used to calculate blood pressure from the optical and electrical waveforms. These are described in the following co-pending patent applications, the contents of which are incorporated herein by reference: 1) CUFFLESS BLOOD-PRESSURE MONITOR AND ACCOMPANYING WIRELESS, INTERNET-BASED SYSTEM (U.S. Ser. No. 10/709,015; filed Apr. 7, 2004); 2) CUFFLESS SYSTEM FOR MEASURING BLOOD PRESSURE (U.S. Ser. No. 10/709,014; filed Apr. 7, 2004); 3) CUFFLESS BLOOD PRESSURE MONITOR AND ACCOMPANYING WEB SERVICES INTERFACE (U.S. Ser. No. 10/810,237; filed Mar. 26, 2004); 4) CUFFLESS BLOOD PRESSURE MONITOR AND ACCOMPANYING WIRELESS MOBILE DEVICE (U.S. Ser. No. 10/967,511; filed Oct. 18, 2004); 5) BLOOD PRESSURE MONITORING DEVICE FEATURING A CALIBRATION-BASED ANALYSIS (U.S. Ser. No. 10/967,610; filed Oct. 18, 2004); 6) PERSONAL COMPUTER-BASED VITAL SIGN MONITOR (U.S. Ser. No. 10/906,342; filed Feb. 15, 2005); 7) PATCH SENSOR FOR MEASURING BLOOD PRESSURE WITHOUT A CUFF (U.S. Ser. No. 10/906,315; filed Feb. 14, 2005); 8) PATCH SENSOR FOR MEASURING VITAL SIGNS (U.S. Ser. No. 11/160,957; filed Jul. 18, 2005); 9) WIRELESS, INTERNET-BASED SYSTEM FOR MEASURING VITAL SIGNS FROM A PLURALITY OF PATIENTS IN A HOSPITAL OR MEDICAL CLINIC (U.S. Ser. No. 11/162,719; filed Sep. 9, 2005); 10) HAND-HELD MONITOR FOR MEASURING VITAL SIGNS (U.S. Ser. No. 11/162,742; filed Sep. 21, 2005); 11) SYSTEM FOR MEASURING VITAL SIGNS USING AN OPTICAL MODULE FEATURING A GREEN LIGHT SOURCE (U.S. Ser. No. 11/307,375; filed Feb. 3, 2006); 12) BILATERAL DEVICE, SYSTEM AND METHOD FOR MONITORING VITAL SIGNS (U.S. Ser. No. 11/420,281; filed May 25, 2006); 13) SYSTEM FOR MEASURING VITAL SIGNS USING BILATERAL PULSE TRANSIT TIME (U.S. Ser. No. 11/420,652; filed May 26, 2006); 14) BLOOD PRESSURE MONITOR (U.S. Ser. No. 11/530,076; filed Sep. 8, 2006); and 15) TWO-PART PATCH SENSOR FOR MONITORING VITAL SIGNS (U.S. Ser. No. 11/558,538; filed Nov. 10, 2006).

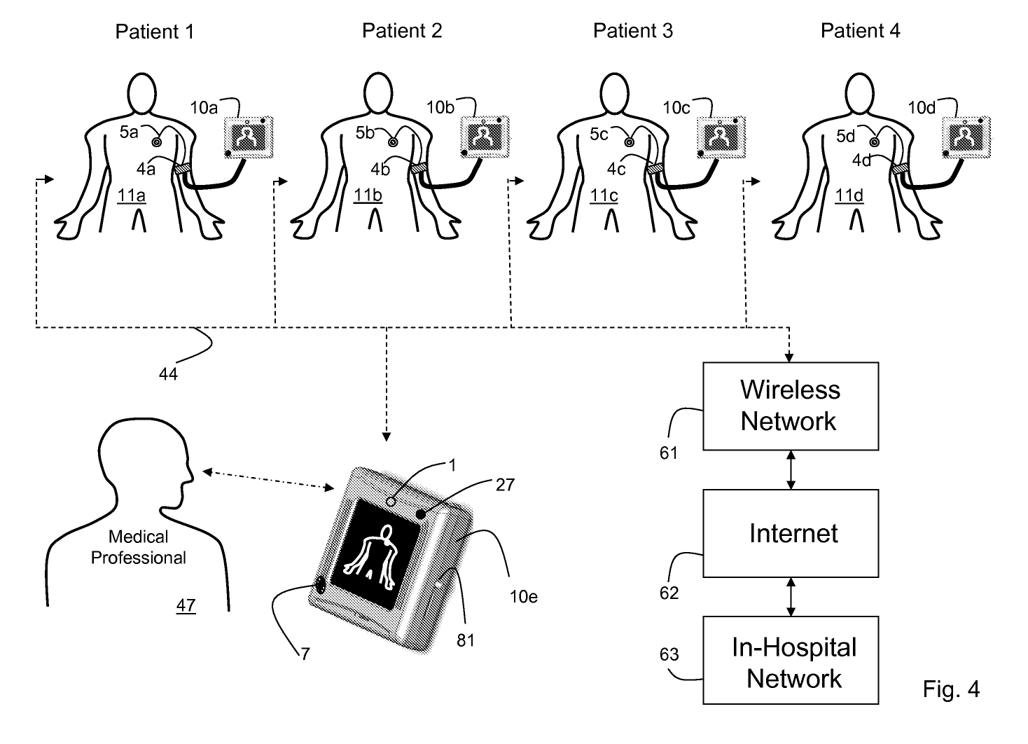

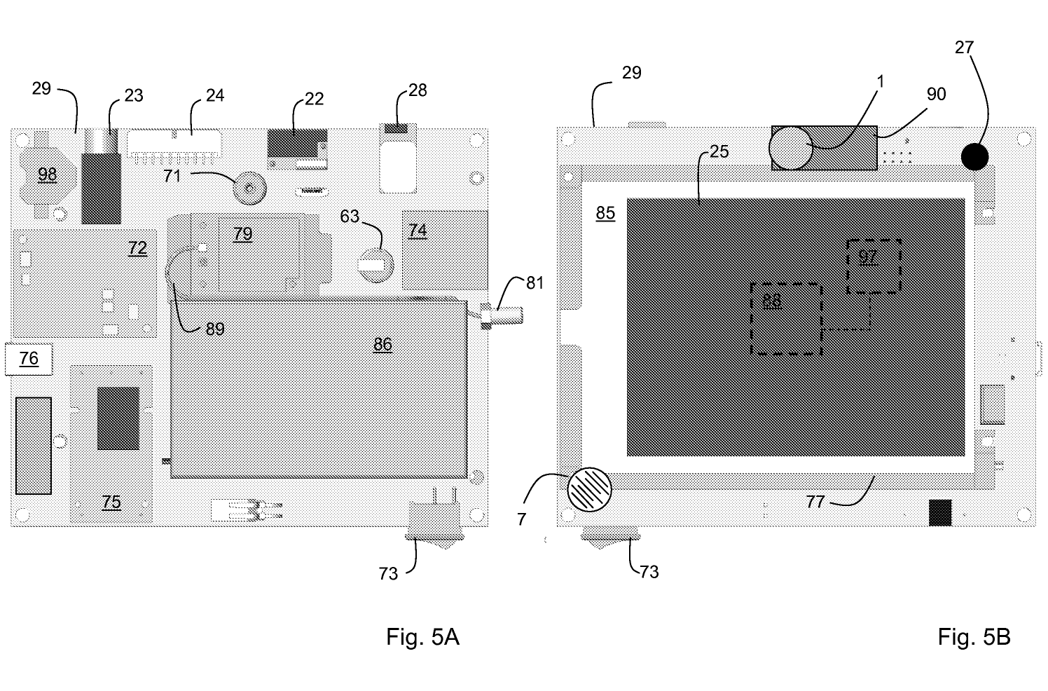

FIG. 4 shows how a first monitor 10e associated with a medical professional 47 operates in a hospital environment to collect vital sign information from four separate monitors 10a-d, each associated with a separate pad sensor 4a-d and electrode 5a-d, and patient 11a-d. Each patient 11a-d, for example, is typically located in a unique hospital room. The medical professional 47 uses the first monitor 10e to make ‘virtual rounds’ by capturing video, audio, and vital sign information from each patient 11a-d. During this process, the digital video camera 1, digital audio microphone 27, and speaker 7 from the first monitor 10e captures video and audio information from the medical professional 47 and transmits this to the monitors 10a-d associated with each patient 11a-d. Likewise, the four separate monitors 10a-d capture video and audio information, along with vital signs, from the four patients 11a-d and transmit this information to the medical professional's monitor 10e. The monitors 10a-e typically communicate through a short-range wireless connection 44 (using, e.g. a Bluetooth® or 802.11-based transceiver), described further in FIGS. 5A and 5B. Once vital sign information is collected from each patient 11a-d, the device 10e formats the data accordingly and sends it using an antenna 81 through a nation-wide wireless network 61 to a computer system on the Internet 62. The computer system then sends the information through the Internet 62 to an in-hospital network 63 (using, e.g., a frame-relay circuit or VPN). From there, the information is associated with the patient's medical records, and can be accessed at a later time by the medical professional.

FIGS. 5A and 5B show a circuit board 29 mounted within the monitor for measuring vital signs and rendering video images as described above. A rechargeable lithium-ion battery 86 (manufacturer: Varta Microbattery; part number: 3P/PLF 503562 C PCM W) powers each of the circuit elements and is controlled by a conventional on/off switch 73. A smaller back-up battery 98 is used to power volatile memory components. All compiled computer code that controls the monitor's various functions runs on a high-end microprocessor 88, typically an ARM 9 (manufacturer: Atmel; part number: AT91SAM9261-CJ), that is typically a ‘ball grid array’ package mounted underneath an LCD display 85. Before being processed by the microprocessor 88, analog signals from the optical and electrical sensors pass through a connector 24 to the analog-to-digital converter 97, which is typically a separate integrated circuit (manufacturer: Texas Instruments; part number: ADS8344NB) that digitizes the waveforms with 16-bit resolution. Such high resolution is typically required to adequately process the optical and electrical waveforms, as described in more detail below. The microprocessor 88 also controls a pulse oximetry circuit 72 including a connector (not shown in the figure) that connects to an external pulse oximetry finger sensor. To measure temperature, a probe containing a temperature-sensitive sensor (e.g. a thermistor) connects through a stereo jack-type connector 24, which in turn connects to the analog-to-digital converter 97. During operation, the temperature-sensitive sensor generates an analog voltage that varies with the temperature sensed by the probe. The analog voltage passes to the analog-to-digital converter 97, where it is digitized and sent to the microprocessor 88 for comparison to a pre-determined look-up table stored in memory. The look-up table correlates the voltage measured by the temperature probe to an actual temperature.

After calculating vital signs, the microprocessor 88 displays them on the LCD 85 (manufacturer: EDT; part number: ER05700NJ6*B2), which additionally includes a touch panel 25 on its outer surface, and a backlight 77 underneath. An LCD control circuit 75 includes a high-voltage power supply that powers the backlight, and an LCD controller that processes signals from the touch panel 25 to determine which coordinate of the LCD 85 was contacted with the stylus. The microprocessor 88 runs software that correlates coordinates generated by the LCD controller with a particular icon and ultimately to software functions coded into the microprocessor 88.

Information can be transferred from the monitor to an external device using both wired and wireless methods. For wired transfer of information, the circuit board 29 includes a universal serial bus (USB) connector 76 that connects directly to another device (e.g. a personal computer), and a removable SD flash memory card 74 that functions as a removable storage medium for large amounts (e.g., 1 GByte and larger) of information. For wireless transfer of information, the circuit board 29 includes a short-range Bluetooth® transceiver 28 that sends information over a range of up to 30 meters (manufacturer: BlueRadios; part number: BR-C40A). The Bluetooth® transceiver 28 can be replaced with a wireless transceiver that operates on a wireless local-area network, such as a WiFi® transceiver (manufacturer: DPac; part number: WLNB-AN-DP101). For long-range wireless transfer of information, the circuit board 29 includes a CDMA modem 79 (manufacturer: Wavecom; part number: Wismo Quik WAV Q2438F) that connects through a thin, coaxial cable 89 to an external antenna 81. The CDMA modem 79 can be replaced with a comparable long-range modem, such as one that operates on a GSM or IDEN network.

The circuit board 29 includes a barcode scanner 22 (manufacturer: Symbol; part number: ED-95S-I100R) that can easily be pointed at a patient to scan their wrist-worn barcode. The barcode scanner 22 typically has a range of about 5-10 cm. Typically the barcode scanner 22 includes an internal, small-scale microprocessor that automatically decodes the barcode and sends it to the microprocessor 88 through a serial port for additional processing.

A small-scale, noise-making piezoelectric beeper 71 connects to the microprocessor 88 and sounds an alarm when a vital sign value exceeds a pre-programmed level. A small-scale backup battery 63 powers a clock (not shown in the figure) that sends a time/date stamp to the microprocessor 88, which then includes it with each stored data file.

The digital video camera 1 (e.g., Firewire Camera) and digital video frame capture circuit board 90 are positioned in the top-center of the circuit board 29. A digital audio microphone 27 and speaker 7 are positioned, respectively, on the top-right and bottom-left portion of the circuit board 29. Once recorded using the video camera 1 and microphone 27, video and audio information are digitally encoded and relayed to the microprocessor 88 for broadcast through short-range Bluetooth® transceiver 28 to another monitor 10a-e, stored on the SD flash memory card 74, and/or sent to an external database.

FIG. 6 shows a schematic drawing of a software architecture 180 that runs on the above-described microprocessor. The software architecture 180 allows the patient or healthcare professional to operate the GUI 162 to measure vital signs and operate all the electrical components shown in FIGS. 5A and 5B. The software architecture 180 is based on an operating system 160 called the μC/OS-II (vendor: Micrium) which is loaded onto the microprocessor and operates in conjunction with software libraries (vendor: Micrium) for the GUI 162. Using the digital microphone and video camera, the patient or healthcare professional records raw audio and video using an audio/video capture 165 module. The audio/video capture module 165 is allocated to the microprocessor 88, described above, using ATMEL software layer 167 to process and store the captured data. The audio and video data, in turn, are encoded using the audio and video encoder 161 and allocated to the event processor 172 for recall using the GUI 162 or distribution over a network using a network module 163. A USB 166 library (vendor: Micrium) operates the transfer of stored patient vital signs data through a USB cable to external devices. A Microsoft Windows™-compatible FAT32 embedded file management system database (FS/DB) 168 is a read-write information-allocation library that stores allocated patient information, audio and video capture and allows retrieval of information through the GUI 162. These libraries are compiled along with proprietary data acquisition code 164 library that collects digitized waveforms and temperature readings from the analog-to-digital converter and stores them into RAM. The event processor 172 is coded using the Quantum Framework (QF) concurrent state machine framework (vendor: Quantum Leaps). This allows each of the write-to libraries for the GUI 162, USB 166, file system 168, and data acquisition 162 to be implemented as finite state machines (‘FSM’). This process is described in detail in the co-pending patent application ‘HAND-HELD VITAL SIGNS MONITOR’, U.S. Ser. No. 11/470,708, filed Sep. 7, 2006, the contents of which are incorporate herein by reference.

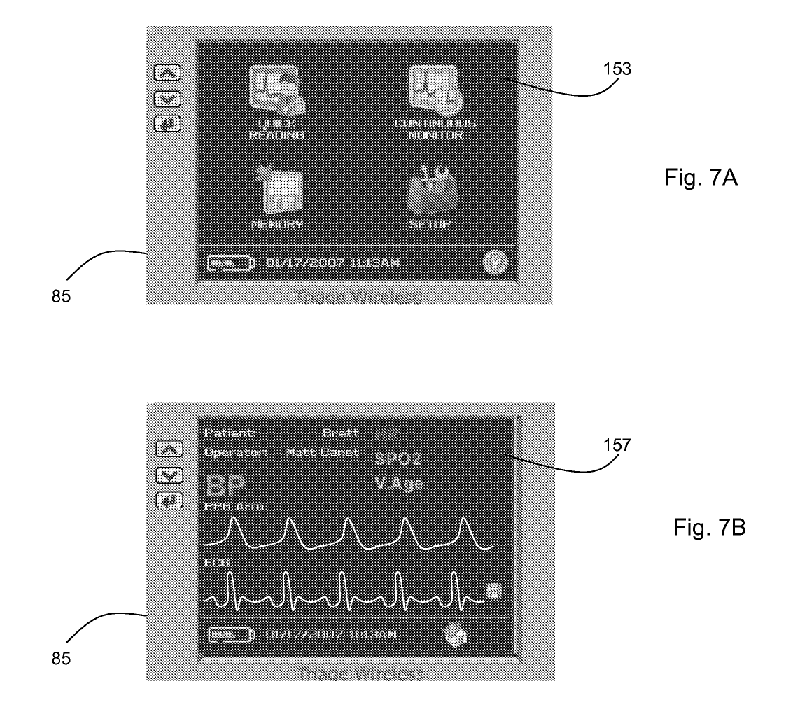

FIGS. 7A and 7B show screen captures of first and second software interfaces 153, 157 within the GUI that run on the LCD 85. Referring to FIG. 7A, the first software interface 153 functions as a ‘home page’ and includes a series of icons that perform different functions when contacted through the touch panel. The home page includes icons for ‘quick reading’, which takes the user directly to a measurement screen similar to that shown in the second software interface 157, and ‘continuous monitor’, which allows the user to enter patient information (e.g. the patient's name and biometric information) before taking a continuous measurement. Information for the continuous measurement is entered either directly using a soft, on-screen QWERTY touch-keyboard, or by using the barcode scanner. Device settings for the continuous measurement, e.g. alarm values for each vital sign and periodicity of measurements, are also entered after clicking the ‘continuous monitor’ icon. The home page additionally includes a ‘setup’ icon that allows the user to enter their information through either the soft keyboard or barcode scanner. Information can be stored and recalled from memory using the ‘memory’ icon. The ‘?’ icon renders graphical help pages for each of the above-mentioned functions.

The second software interface 157 shown in FIG. 7B is rendered after the user initiates the ‘quick reading’ icon in first software interface 153 of FIG. 6A. This interface shows the patient's name (entered using either the soft keyboard or barcode scanner) and values for their systolic and diastolic blood pressure, heart rate, pulse oximetry, and temperature. The values for these vital signs are typically updated every few seconds. In this case the second software interface 157 shows an optical waveform measured with one of the optical sensors, and an electrical signal measured by the electrical sensors. These waveforms are continually updated on the LCD 85 while the sensor is attached to the patient.

Both the first 153 and second 157 software interfaces 157 include smaller icons near a bottom portion of the LCD 85 that correspond to the date, time, and remaining battery life. The ‘save’ icon (indicated by an image of a floppy disk) saves all the current vital sign and waveform information displayed measured by the monitor to an on-board memory, while the ‘home’ icon (indicated by an image of a house) renders the first software interface 153 shown in FIG. 7A.

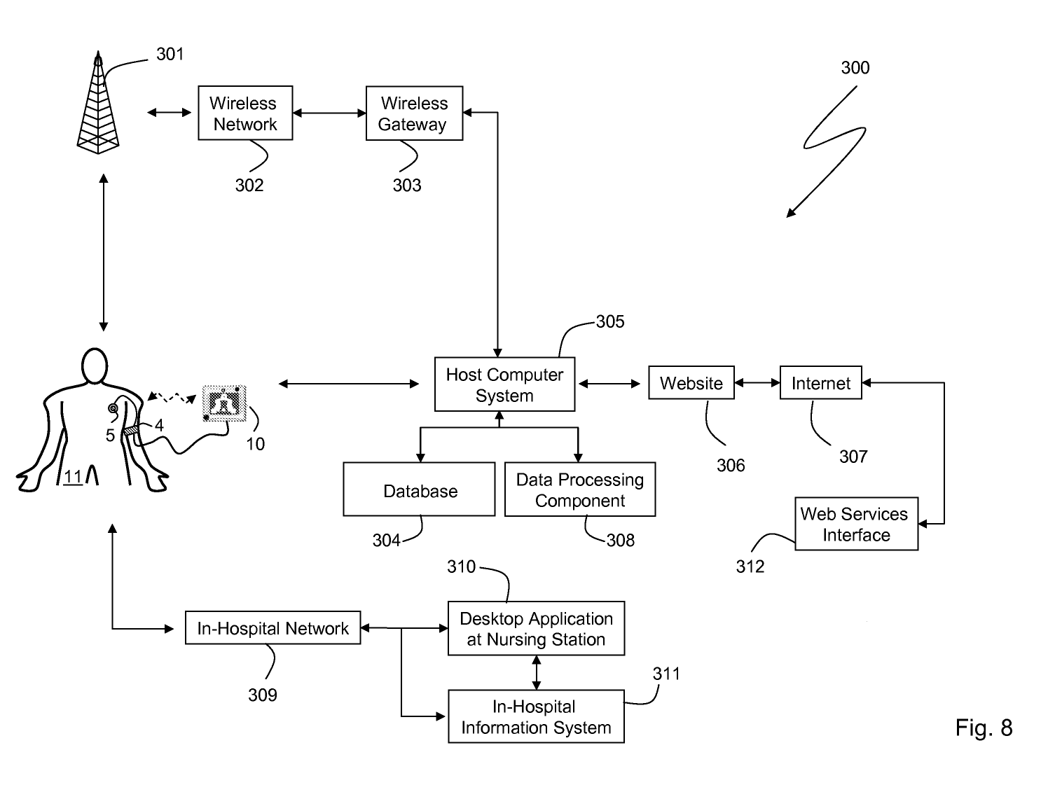

FIG. 8 shows an example of a computer system 300 that operates in concert with the monitor 10 and sensors 4, 5 to measure and send information from a patient 11 to an host computer system 305, and from there to an in-hospital information system 311. When the patient is ambulatory, the monitor 10 can be programmed to send information to a website 306 hosted on the Internet. For example, using an internal wireless modem, the monitor 10 sends vital signs and video/audio information through a series of towers 301 in a nation-wide wireless network 302 to a wireless gateway 303 that ultimately connects to a host computer system 305. The host computer system 305 includes a database 304 and a data-processing component 308 for, respectively, storing and analyzing data sent from the monitor 10. The host computer system 305, for example, may include multiple computers, software systems, and other signal-processing and switching equipment, such as routers and digital signal processors. The wireless gateway 303 preferably connects to the wireless network 302 using a TCP/IP-based connection, or with a dedicated, digital leased line (e.g., a VPN, frame-relay circuit or digital line running an X.25 or other protocols). The host computer system 305 also hosts the web site 306 using conventional computer hardware (e.g. computer servers for both a database and the web site) and software (e.g., web server, application server, and database software).

To view information remotely, the patient or medical professional can access a user interface hosted on the web site 306 through the Internet 307 from a secondary computer system such as an Internet-accessible home computer. The computer system 300 may also include a call center, typically staffed with medical professionals such as doctors, nurses, or nurse practitioners, whom access a care-provider interface hosted on the same website 306.

Alternatively, when the patient is in the hospital, the monitor can be programmed to send information to an in-hospital information system 311 (e.g., a system for electronic medical records). In this case, the monitor 10 sends information through an in-hospital wireless network 309 (e.g., an internal WiFi® network) that connects to a desktop application running on a central nursing station 310. This desktop application 310 can then connect to an in-hospital information system 311. These two applications 310, 311, in turn, can additionally connect with each other. Alternatively, the in-hospital wireless network 309 may be a network operating, e.g. a Bluetooth®, 802.11a, 802.11b, 802.1g, 802.15.4, or ‘mesh network’ wireless protocols that connects directly to the in-hospital information system 311. In these embodiments, a nurse or other medical professional at a central nursing station can quickly view the vital signs of the patient using a simple computer interface.

Other embodiments are also within the scope of the invention. For example, software configurations other than those described above can be run on the monitor to give it a PDA-like functionality. These include, for example, Micro C OS®, Linux®, Microsoft Windows®, embOS, VxWorks, SymbianOS, QNX, OSE, BSD and its variants, e.g. FreeDOS, FreeRTOX, LynxOS, or eCOS and other embedded operating systems. In other embodiments, the monitor can connect to an Internet-accessible website to download content, e.g. calibrations, text messages, and information describing medications, from an associated website. As described above, the monitor 10 can connect to the website using both wired (e.g. USB port) or wireless (e.g. short or long-range wireless transceivers) means.

The above-described monitor may be used for in-home monitoring. In this case, the patient may video conference with a healthcare professional (i.e. physician, nurse, or pharmacist) from the comfort of their home or while traveling using the wireless or Internet-based technology, described above. The health care professional may access real-time vital signs information or vital signs information that has been stored over a period of time (e.g., an hour, day, week, or up to months).

Still other embodiments are within the scope of the following claims.

I claim as my invention:

1. A device for monitoring a patient's vital signs, comprising:
a sensor component that attaches to the patient and comprises an optical sensor and an electrical sensor that measure, respectively, a first and second signal;
an analog-to-digital converter configured to convert the first signal and second signal into, respectively, a first digital signal and a second digital signal;
a control component comprising:
a CPU configured to operate an algorithm that generates a blood pressure value by processing with an algorithm the first digital signal and second digital signal,
a display element,
a graphical user interface generated by computer code operating on the CPU and configured to render on the display element the blood pressure value, and,
a software component that renders video images on the display element.

2. The device of claim 1, wherein the control component further comprises a digital camera.

3. The device of claim 1, wherein the control component further comprises a microphone.

4. The device of claim 1, wherein the control component further comprises a touch panel connected to the display element.

5. The device of claim 4, wherein the control component further comprises a touch panel controller in electrical communication with the CPU and the touch panel.

6. The device of claim 4, wherein the graphical user interface further comprises a plurality of icons, each corresponding to a different operation on the device.

7. The device of claim 6, wherein the CPU comprises compiled computer code configured to render video images when an icon is addressed through the touch panel.

8. The device of claim 1, wherein the compiled computer code further comprises a video driver.

9. The device of claim 6, wherein the CPU comprises compiled computer code configured to play audio information when an icon is addressed through the touch panel.

10. The device of claim 9, wherein the compiled computer code further comprises an audio driver.

11. The device of claim 1, wherein the control component further comprises a wireless modem.

12. The device of claim 11, wherein the control component further comprises a wireless modem in electrical communication with the CPU, the wireless modem configured to receive video information over a wireless interface and provide the video information to the CPU.

13. The device of claim 11, wherein the control component further comprises a wireless modem configured to operate on a wide-area wireless network.

14. The device of claim 13, wherein the control component further comprises a wireless modem configured to operate on a CDMA, GSM, or IDEN wireless network.

15. The device of claim 11, wherein the control component further comprises a wireless modem configured to operate on a local-area wireless network.

16. The device of claim 15, wherein the control component further comprises a wireless modem configured to operate on a local-area network based on a protocol selected from: 802.11, 802.15, 802.15.4.

17. A device for monitoring a patient's vital signs, comprising:
a body-worn component that attaches to the patient and comprises:
a sensor that measures at least one vital sign,
a microprocessor that receives and processes the at least one vital sign from the sensor, and
a first short-range wireless transceiver in electrical communication with the microprocessor that wirelessly transmits the at least one vital sign;
a control component comprising:
a second short-range wireless transceiver that receives the at least one vital sign from the first short-range wireless transceiver,
a CPU configured to receive and process the at least one vital sign,
a display element,
a graphical user interface generated by computer code operating on the CPU and configured to render on the display element the at least one vital sign, and,
a software component that renders video images on the display element.

18. The device of claim 17, further comprising a digital camera.

19. The device of claim 17, further comprising a microphone.

20. The device of claim 17, further comprising a touch panel connected to the display element.

21. The device of claim 20, further comprising a touch panel controller in electrical communication with the CPU and the touch panel.

22. The device of claim 20, wherein the graphical user interface further comprises a plurality of icons, each corresponding to a different operation on the device.

23. The device of claim 22, wherein the CPU comprises compiled computer code configured to render video images when an icon is addressed through the touch panel.

24. The device of claim 17, wherein the compiled computer code further comprises a video driver.

25. The device of claim 22, wherein the CPU comprises compiled computer code configured to play audio information when an icon is addressed through the touch panel.

26. The device of claim 25, wherein the compiled computer code further comprises an audio driver.

27. A device for monitoring a patient's vital signs, comprising:
a body-worn component that attaches to the patient and comprises:
a sensor that measures at least one vital sign,
a microprocessor that receives and processes the at least one vital sign from the sensor, and
a first short-range wireless transceiver in electrical communication with the microprocessor that wirelessly transmits the at least one vital sign;
a control component comprising:
a second short-range wireless transceiver that receives the at least one vital sign from the first short-range wireless transceiver,
a CPU configured to receive and process the at least one vital sign,
a display element,
a graphical user interface generated by computer code operating on the CPU and comprising a first and second icon, the graphical user interface configured to render on the display element the at least one vital sign when the first icon is addressed, and,
a software component that renders real-time video and audio information on the display element when the second icon comprised by the graphical user interface is addressed.
